(19) United States
(12) Patent Application Publication
(10) Pub. No.: US 2014/0123876 A1
(43) Pub. Date: May 8, 2014

(54) HYDROXY COMPOUNDS OBTAINED FROM 2,2,3,3-TETRAFLUOROOXETHANE AND DERIVATIVES THEREOF

(71) Applicants: Simonetta Antonella Fontana, Milano (IT); Claudio Adolfo Pietro Tonelli, Paderno D'adda (IT); Piero Gavezotti, Milano (IT)

(72) Inventors: Simonetta Antonella Fontana, Milano (IT); Claudio Adolfo Pietro Tonelli, Paderno D'adda (IT); Piero Gavezotti, Milano (IT)

(73) Assignee: SOLVAY SPECIALTY POLYMERS ITALY S.P.A., Bollate, MI (IT)

(21) Appl. No.: 14/124,999

(22) PCT Filed: Jun. 1, 2012
(86) PCT No.: PCT/EP2012/060379

§ 371 Date: Dec. 9, 2013

(30) Foreign Application Priority Data
Jun. 10, 2011 (EP) 11169458.4

(51) Int. Cl. C07C 43/13; C07C 41/02; C09D 7/12; C07F 9/09

(52) U.S. Cl. CPC C07C 43/137; C07F 9/091; C07C 41/02; C09D 7/1233
USPC 106/287.26; 568/615; 558/92; 558/186; 106/287.23

(57) ABSTRACT

The invention relates to a hydroxy compound of formula (I) wherein n is an integer equal to or higher than 1 as an intermediate for the preparation of compounds of formulae (IIa) and (IIIb) as defined in the specification that can be used as water and oil resistance agents. The invention further relates to water and oil resistance compositions containing compounds (IIa) and (IIb).

F(CH₂CF₂CF₂O)ₙCH₂CF₂CH₂OH (I)

### CROSS REFERENCE TO RELATED APPLICATION

This application claims priority to European patent application 11169458.4, filed on Jun. 10, 2011, the whole content of which is herein incorporated for all purposes.

### TECHNICAL FIELD

The present invention relates to hydroxy compounds and derivatives thereof obtained through anionic ring-opening reaction of 2,2,3,3-tetrafluorooxethane initiated by metal fluorides followed by reduction and derivatization of the COF end groups.

### BACKGROUND ART

Ring-opening reactions of 2,2,3,3-tetrafluorooxethane initiated by metal fluorides are known, for example, from U.S. Pat. No. 4,719,052 and U.S. Pat. No. 4,845,268, both to Daikin Industries Ltd. The former discloses the ring-opening reaction of 2,2,3,3-tetrafluorooxethane initiated by alkali metal fluorides (such as sodium fluoride, potassium fluoride and cesium fluoride) to provide derivatives of 2,2-difluoropropionic acid, which are taught to be useful as intermediates for medicines, agricultural chemicals and strong acid catalysts.

The latter discloses the ring-opening polymerization of 2,2,3,3-tetrafluorooxethane initiated by alkali metal halides, such as potassium fluoride, potassium iodide, potassium bromide and cesium fluoride, to provide compounds of formula A-(CH₂CF₂CF₂O)ₐ-CH₂-CF₂COF, wherein A is halogen and a is an integer of not less than 1, which can subsequently converted into a corresponding acid, ester or amide by conventional methods.

Neither U.S. Pat. No. 4,719,052 nor U.S. Pat. No. 4,845,268 teaches or suggests to reduce the COF terminal groups in such a way as to obtain hydroxy compounds to be used as intermediates for further functional derivatives.

U.S. Pat. No. 2,876,247 Ato Olin Mathieson Chemical Corporation discloses polymeric polyfluoroalkyl phosphonitrilates of the formula:

[NP[OCH₂(CF₂)ₘ•Y]₂]ₙ

wherein Y is selected from the group consisting of hydrogen and fluorine, m is a whole number from 1 to 20 and n is a whole number from 3 to 7. This patent document neither discloses nor suggest phosphonitrilates bearing other chains than polyfluoroalkyl chains.

### SUMMARY OF THE INVENTION

It has now been found that fluoroacyl compounds obtained through ring-opening reaction of 2,2,3,3-tetrafluorooxethane initiated by metal fluorides can be conveniently reduced to the corresponding hydroxy compounds, which can be used as intermediates for the preparation of derivatives endowed with hydrophobic or oilophobic properties.

Thus, according to a first aspect, the present invention relates to a hydroxy compound of formula (I)

F(CH₂CF₂CF₂O)ₙCH₂CF₂CH₂OH (I)

wherein n is an integer equal to or higher than 1, preferably an integer ranging from 1 to 100, more preferably from 1 to 50 and even more preferably from 5 to 15.

According to a second aspect, the invention relates to derivatives of the hydroxy compound of formula (I).

In a first embodiment, the derivatives are compounds complying with formula (IIa)

F(CH₂CF₂CF₂O)ₙCH₂CF₂CH₂OR¹ (IIa)

wherein n is as defined above and R¹ is a (CHR²CHR³O)ₘ-R⁴ group, in which:

R² and R³ are both hydrogen or one is hydrogen and the other is methyl;

m is 0 or an integer equal to or higher than 1;

R⁴ is selected from:
hydrogen, with the proviso that, when m is 0, R⁴ is not hydrogen;
a straight or branched, saturated or unsaturated C₁-C₃₆ hydrocarbon chain; a C₅-C₁₄ cycloaliphatic or a C₁-C₁₀ aliphatic-C₅-C₁₄ cycloaliphatic group; a C₆-C₁₄ aromatic or a C₁-C₁₀ aliphatic-C₆-C₁₄ aromatic group;
a P(O)R⁵R⁶ group, in which R⁵ and R⁶ are, independently of each other, selected from hydroxy and —O⁻X⁺ groups, in which X⁺ is selected from from Li⁺, Na⁺, K⁺, (NH₃R′)⁺, (NH₂R′R″)⁺ and (NHR′R″R′″)⁺ wherein R is H or a linear or branched C₁-C₂₂ alkyl group optionally containing one or more —OH groups, and R′, R″ and R′″, equal to or different from each other, are linear or branched C₁-C₂₂ alkyl groups optionally containing one or more —OH groups or optionally linked to each other to form N-heterocyclic groups;
a COR⁷ group, in which R⁷ is a straight or branched, saturated or unsaturated C₁-C₃₆ hydrocarbon chain, preferably a —CR_{H}═CH₂ chain, wherein R_{H} is hydrogen or a straight or branched, saturated or unsaturated C₁-C₃₄ hydrocarbon chain, or R⁷ is a C₅-C₁₄ cycloaliphatic or a C₁-C₁₀ aliphatic-C₅-C₁₄ cycloaliphatic group; a C₆-C₁₄ aromatic or a C₁-C₁₀ aliphatic-C₆-C₁₄ aromatic group;
a CONHR⁸ group, in which R⁸ is the same as R⁷ or a COR⁷ group, in which R⁷ is as defined above;
a CO—R^{A}—CR_{H}═CH₂ group, wherein R_{H} is hydrogen or a straight or branched, saturated or unsaturated C₁-C₃₄ hydrocarbon chain and R^{A} is selected from the group consisting of
(j) —NH—R^{B}—O—CO—
(jj) —NH—R^{B}—NHCOO—R^{B}—OCO—
(jjj) —RB—O—CO—
RB being a divalent group selected from a C₁-C₁₀ aliphatic group, a C₅-C₁₄ cycloaliphatic group a C₆-C₁₄ aromatic or a C_{1-C}₁₀ aliphatic-C₆-C₁₄ aromatic group.

Preferred compounds of formula (IIa) are those in which n ranges from 1 to 100, preferably from 1 to 50, even more preferably from 5 to 15.

Preferred compounds of formula (IIa) are also those in which m ranges from 0 to 10, more preferably from 0 to 3; even more preferably, m is 1.

Preferred compounds of formula (IIa) are also those in which and R² and R³ are both hydrogen.

Among compounds in which R⁴ is hydrogen or a hydrocarbon chain, those in which m ranges from 0 to 10, preferably from 0 to 3 and R² and R³ are both hydrogen are preferred; in these compounds, m is more preferably 1. An example of particularly preferred compound is the one in which n is 6, R² and R³ are both hydrogen, m is 1 and R⁴ is hydrogen.

Among compounds of formula (IIa) in which R⁴ is a P(O)R⁵R⁶ group, those in which m ranges from 0 to 10, preferably from 0 to 3, R² and R³ are both hydrogen and R⁵ and R⁶ are both hydroxy groups are preferred; in these compounds, m is more preferably 1. An example of a particularly preferred compound is the one in which n is 6, R² and R³ are both hydrogen, m is 1 and R⁵ and R⁶ are both hydroxy groups.

Among compounds of formula (IIa) in which R⁴ is a COR⁷ group, those in which R⁷ is a —CR_{H}═CH₂ chain are preferred, the —C(CH₃)═CH₂ chain being particularly preferred. Particularly preferred are compounds in which n ranges from 5 to 15, R² and R³ are both hydrogen, m ranges from 0 to 10, more preferably from 0 to 3 and R⁷ is a —C(CH₃)═CH₂ chain; in these compounds, m is even more preferably 1.

Among compounds of formula (IIa) in which R⁴ is a CONHR⁸ group, those in which R⁸ is a —CR_{H}═CH₂ chain or in which R⁸ is a COR⁷ group in which R⁷ is a —CR_{H}═CH₂ chain are preferred, the —C(CH₃)═CH₂ chain being particularly preferred. Particularly preferred are compounds in which n ranges from 5 to 15, R² and R³ are both hydrogen, m ranges from 0 to 10, more preferably from 0 to 3 and R⁷ is a —C(CH₃)═CH₂ chain; even more preferably, m is 1.

Among compounds of formula (IIa) in which R⁴ is a CO—R^{A}—CR_{H}═CH₂ group, particularly preferred are those in which R⁴ is CO—R^{A}—C(CH₃)═CH₂ in which R^{A} is a group selected from (j) to (jjj) as defined above. Particularly preferred are the compounds in which n ranges from 5 to 15, R² and R³ are both hydrogen, m ranges from 0 to 10, more preferably from 0 to 3 and R⁴ is a CO—R^{A}—C(CH₃)═CH₂; in these compounds, m is even more preferably 1.

In a second embodiment, the derivatives are phosphazene compounds complying with general formula (IIb)

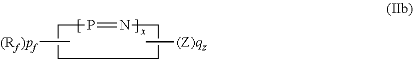

wherein:

R_{f} is a group of formula F(CH₂CF₂CF₂O)ₙCH₂CF₂CH₂O(CHR²CHR³O)ₘ in which R², R³, n and m are as defined above;

Z is a polar group of formula —O⁻M⁺, wherein M is selected from hydrogen, a monovalent metal, preferably an alkali metal selected from Li, Na, K, an ammonium radical selected from NRₐR_{b}R_{c}R_{d}, wherein each of Rₐ, R_{b}, R_{c} and R_{d} is, independently, a hydrogen atom or a C₁-C₂₂ hydrocarbon group, optionally fluorinated, or a polar group of formula —O⁻)₂ M′²⁺, wherein M′ is a divalent metal, preferably an alkaline earth metal selected from Ca and Mg;

x is 3 or 4, with the proviso that:

when x is 3, p_{f} is an integer from 1 to 6, q_{z} is 0 or an integer from 1 to 5 and p_{f}+q_{z} is 6;

when x is 4, pf is an integer from 1 to 8, q_{z} is 0 or an integer from 1 to 7 and p_{f}+q_{z} is 8.

Preferred compounds of formula (IIb) are those in which x is 3, R_{f} is a group of formula F(CH₂CF₂CF₂O)ₙCH₂CF₂CH₂O(CHR²CHR³O)ₘ- in which n is an integer from 5 to 15, m is 0 and and p_{f} is 6; however, compounds (IIb) in which p_{f} is 6 can be advantageously used in admixture with compounds in which p_{f} is equal to or lower than 5 and q_{z} is equal to or higher than 1.

A particularly preferred compound of formula (IIb) is that in which x is 3, R_{f} is a group of formula F(CH₂CF₂CF₂O)₆CH₂CF₂CH₂O— and p_{f} is 6; most preferably, this compound is used in admixture with a partially salified corresponding compound, such as the compound in which p_{f} is 5 and Z is a polar group of formula —O⁻)₂Ca²⁺.

The hydroxy compounds of formula (I) can be prepared through a process which comprises:

a) ring-opening reaction of 2,2,3,3-tetrafluorooxethane (III)

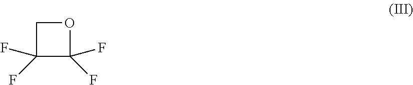

with a organic or inorganic fluoride, to provide a fluoro acyl compound of formula (IV)

F(CH₂CF₂CF₂)ₙCH₂CF₂COF (IV)

wherein n is as defined above and

b) reducing the fluoroacyl compound of formula (IV) to a hydroxy compound of formula (I).

For the purposes of the present invention, the expression “ring-opening reaction” means a reaction whereby 2,2,3,3-tetrafluorooxethane undergoes oligo- or polymerization.

The ring-opening reaction of step a) is carried out in an aprotic solvent, typically acetonitrile or a glycol dialkyl ether; among glycol dialkyl ethers, diglyme and tetraglyme are preferred. The reaction is carried out at a temperature ranging from about −30° C. to about +30° C., preferably from about −5° C. to about +10° C., more preferably at about 0° C.

The organic fluoride is usually selected from ammonium fluoride or an alkyl ammonium fluoride, such as tetrabutylammonium fluoride, while the inorganic fluoride is usually selected from lithium fluoride, sodium fluoride, potassium fluoride, calcium fluoride, barium fluoride, magnesium fluoride and cesium fluoride; according to a preferred embodiment, the metal fluoride is cesium fluoride.

The reduction of the fluoroacyl compound of formula (IV) (step b) is preferably carried out by reacting compound (IV) with an alcohol, to provide an ester that is subsequently reduced to the hydroxy compound of formula (I). Non-limiting examples of alcohols are methanol, ethanol, n-propanol, iso-propanol and tert-butanol, ethanol being preferred. The reduction of the ester to the hydroxy compound (I) is carried out with a metal hydride, typically NaBH₄, in an appropriate solvent, which is usually selected from alcohols, typically ethanol, ethers and glycols, like glyme.

It is also possible to reduce compound (IV) by converting it into a corresponding carboxylic acid or into an ester of a carboxylic acid, typically the ethyl ester, and then by reducing the carboxylic acid or carboxy ester under H₂ pressure in the presence of a homogeneous or heterogeneous metal-supported catalysts; preferred catalysts are those based on metals of group VIII of the Periodic Table, preferably Pt, Rh, Ru, more preferably carbon-supported ruthenium. This reduction method is disclosed in U.S. Pat. No. 7,132,574 (SOLVAY SOLEXIS SPA).

The compounds of formulae (IIa) and (IIb) as defined above can be prepared according to methods known to those skilled in the art.

In particular, the compounds of formula (IIa) in which R¹ is (CHR²CHR³O) ₘ—R⁴ wherein m is equal to or higher than 1 can be prepared by reacting the hydroxy compound of formula (I) with ethylene oxide, propylene oxide, ethylene carbonate or propylene carbonate in the presence of an inorganic or organic base catalyst, for example an alkaline or terrous-alkaline hydroxide, or tertiary amines.

The compounds of formula (IIa) in which R¹ is (CHR²CHR³O)ₘ—R⁴ in which R⁴ is a hydrocarbon chain can be prepared by transforming the hydroxy group in a hydroxy compound of formula (I) or in a compound of formula (IIa) in which R¹ is (CHR²CHR³O)ₘ—R⁴ wherein m is equal to or higher than 1 and R⁴ is hydrogen into a leaving group, such as a tosylate, nonaflate or triflate, and by reacting the resulting derivative with a nucleophilic alcoholate. As an alternative, a the hydroxy group in a compound of formula (I) or in a compound of formula (IIa) in which R¹ is (CHR²CHR³O)ₘ—R⁴ wherein m is equal to or higher than 1 and R⁴ is hydrogen can be transformed into an alkoxy group and reacted with an alcohol in which the alkoxy group has been transformed into a suitable leaving group, for instance a tosylate.

The compounds of formula (IIa) in which R¹ is a (CHR²CHR³O)ₘ—P(O)R⁵R⁶ group can be prepared, for instance, by reacting a hydroxy compound of formula (I) or a compound of formula (IIa) in which R¹ is a (CHR²CHR³O)ₘ—R⁴ group wherein m is equal to or higher than 1 and R⁴ is hydrogen with phosphoryl trichloride (POCl₃) in the presence of a base as HCl acceptor, or by reacting a compound of formula (I) or a compound of formula (IIa) in which R¹ is a (CHR²CHR³O)ₘ—R⁴ group wherein m is equal to or higher than 1 and R⁴ is hydrogen with phosphorus pentoxide (P₂O₅), and optionally salifying the resulting derivative.

The compounds of formula (IIa) in which R¹ is a (CHR²CHR³O)ₘ—COR⁷ group can be prepared by reacting a compound of formula (I) or a compound of formula (IIa) in which R¹ is a (CHR²CHR³O)ₘ—R⁴ group wherein m is equal to or higher than 1 and R⁴ is hydrogen with a carboxylic acid R⁷CO₂H or with a reactive derivative thereof; for the purposes of the present description, the expression “reactive derivative” of carboxylic acids is meant to comprise chlorides, bromides, iodides and esters.

The compounds of formula (IIa) in which R¹ is a (CHR²CHR³O)ₘ—CONHR ⁸ group in which R⁸ is a hydrocarbon chain can be prepared by reacting a hydroxy compound of formula (I) or a compound of formula (IIa) in which R ¹ is a (CHR²CHR³O )ₘ—R⁴ group wherein m is equal to or higher than 1 and R⁴ is hydrogen with an isocyanate of formula R⁸—N═C═O. The compounds in which R⁸ is a R⁷CO group can be prepared by reacting a compound of formula (I) or a compound of formula (IIa) in which R¹ is a (CHR²CHR³O )ₘ—R⁴ group wherein m is equal to or higher than 1 and R⁴ is hydrogen with dichlorocarbonate and by reacting the resulting compound with an amide of formula R⁷CONH₂.

The compounds of formula (IIa) in which R¹ is a (CHR²CHR³O )ₘ—CO—RA —CR_{H}═CH₂ group wherein R^{A} is —NH—R^{B}—O—CO— wherein B is as defined above can be prepared by reacting a hydroxy compound of formula (I) or a compound of formula (IIa) in which R¹ is a (CHR²CHR³O )ₘ—R⁴ group wherein m is equal to or higher than 1 and R⁴ is hydrogen with dichlorocarbonate, followed either by reaction with an amine of formula NH ₂—R^{B}—O—CO—CR_{H}═CH₂ or by reaction with an amino alcohol NH₂—R^{B}—OH and a carboxylic acid of formula CH₂═CR_{H}CO₂H or with a reactive derivative thereof.

The compounds of formula (IIa) in which R¹ is a (CHR²CHR³O )ₘ—CO—R^{A}—CR_{H}═CH₂ group wherein R^{A} is NH—R^{B}—NHCOO—R^{B}—OCO— can be prepared by reacting a hydroxy compound of formula (I) with dichlorocarbonate, followed by reaction of a diamine NH₂—R^{B}—NH₂ to provide a compound of formula (V)

F(CH₂CF₂CF₂O)ₙCH₂CF₂CH₂OCONHR^{B}NH₂ (IV)

which can be further reacted with dichlorocarbonate and a diol HO—R^{B}—OH to provide a compound of formula (VI)

F(CH₂CF₂CF₂O)ₙCH₂CF₂CH₂OCONHR^{B}NHCOOR^{B}—OH (VI)

which, by reaction with a carboxylic acid of formula CH₂═CR_{H}CO₂H or with a reactive derivative thereof affords the desired compounds (IIa). For the preparation of the compounds of formula (IIa) in which m is equal to or higher than 1, the hydroxy compounds of formula (I) are first reacted ethylene oxide, propylene oxide, ethylene carbonate or propylene carbonate.

The compounds of formula (IIa) in which R¹ is a (CHR²CHR³O )ₘ—CO—R^{A}—CR_{H}═CH₂ group in which R^{A} is R^{B}—O—CO— can be prepared by reacting a hydroxy compound of formula (I) with an acid of formula CH₂═CH_{R}—R^{A}—CO₂H or with a reactive derivative thereof. As an alternative, the compounds of formula (IIa) can be reacted with a hydroxy acid of formula PO—R^{B}—COOH, wherein P is a hydroxy-protecting group, to provide a compound of formula (VII)

F(CH₂CF₂CF₂O)ₙCH₂CF₂CH₂OCO—RB—OP (VII)

which, after removal of the protecting group and reaction with a carboxylic acid CH₂═CR_{H}-COOH or with a derivative thereof, affords the desired compound (IIa).

The compounds of formula (IIb) can be prepared by reacting a hydroxy compound of formula (I) or a compound of formula (IIa) in which R¹ is a (CHR²CHR³O)ₘR⁴ group, wherein R² and R³ are as defined above, m is equal to or higher than 1 and R⁴ is hydrogen, with hexafluorotriphosphazene or octachlorotetraphosphazene. The above-mentioned compound (I) or (IIa) is usually dissolved in a fluorinated or hydrofluorinated solvent having a boiling point between 20 and 150° C., preferably between 40 and 100° C., such as perfluorobutyltetrahydrofuran and perfluoropropyltetrahydropyran, and hexafluorotriphosphazene or octachlorotetraphosphazene are usually added in the form of an aqueous alkaline solution; the weight ratio between solvent and compound (I) or (IIa) usually ranges between 0.5-10, preferably between 2 and 5. For the preparation of compounds (IIb) in which q_{z} is an integer from 1 to 5 or 1 to 7, the reaction product is salified with an appropriate compound.

The derivatives of formulae (IIa) and (IIb) as defined above are characterised by low surface energy, high chemical resistance and are able to impart to materials and coatings self-cleaning properties and water- and oil-repellence properties; therefore, a further object of the present invention is the use of the compounds of formulae (IIa) and (IIb) as water and oil resistance agents.

Water and oil resistance compositions containing one or more compounds of formulae (IIa) and (IIb) in admixture with additives or vehicles are a still further object of the invention.

Compounds (IIa) and (IIb) and the compositions containing them can be applied to a variety of substrates, such as glass or cellulose substrates, especially those used in packaging applications.

The compounds of formula (IIa) containing an acrylate function can advantageously be used in nanolithography processes for reproducing (nano)patterns using flexible moulds; for this purpose, compounds (IIa) are applied to a patterned template surface and submitted to UV radiations in the presence of a photoinitiator. A non exhaustive list of photoinitiators and their amounts is reported, for example, in EP 2221664 A (SOLVAY SOLEXIS SPA, AMO GMBH).

The compounds of formula (IIa) containing an acrylate function can also be used as additives for conventional non-fluorinated resins, in order to improve their surface properties, in particular self cleaning and chemical resistance.

The following examples illustrate the invention in greater detail without limiting its scope.

Should the disclosure of any patents, patent applications and publications which are herein incorporated by reference be in conflict with the present application to the extent that it may render a term unclear, the present description shall take precedence.

Raw Materials

2,2,3,3-Tetrafluoroxethane was synthesised from tetrafluoroethylene and formaldehyde according to known methods.

All other reagents are commercially available and they were used without purification.

### Example 1

### Synthesis of F(CH₂CF₂CF₂O)₆CH₂CF₂COOEt

A 500 ml, 3-necked flask, kept under nitrogen and equipped with a magnetic stirrer, a thermometer, a condenser and a dropping funnel was charged with 140 ml ethylenglicol dimethyl ether, 32 g of CsF (Mw=152). The obtained suspension was then cooled to 0° C. and 140 g 2,2,3,3-tetrafluorooxethane (Mw=130) and the reaction mixture was let to stir.

After 3 hours, ¹⁹F-NMR analysis of the crude mixture confirmed the complete reaction of the added 2,2,3,3-tetrafluorooxethane by monitoring the absence of its very typical signals (−80 ppm and −120 ppm).

The reaction mixture was then added with 80 g anhydrous ethanol, warmed to room temperature and let to stir for 1 hour in order to isolate the title compound.

The obtained solution was then poured in a separator funnel containing water in order to obtain separate the fluorinated phase, which was subjected to distillation, thereby affording 136 g of F(CH₂CF₂CF₂O)₆CH₂ CF₂COOEt.

### Example 2

### Synthesis of F(CH₂CF2CF₂O)₆CH₂CF₂CH₂OH

A 250 ml, 3-necked flask, kept under nitrogen and equipped with a magnetic stirrer, a thermometer, a condenser and a dropping funnel was charged with 100 ml anhydrous ethanol and 4 g NaBH₄ (Mw=40). The resulting solution was then cooled to 5° C. and 80 g F(CH₂CF₂CF₂ O)₆CH₂ CF₂COOEt (Mw=917), obtained according to example 1, was slowly added maintaining the temperature below 10° C.

The conversion of the reaction was checked by ¹⁹F-NMR analysis monitoring the disappearance at −114 ppm of the preterminal CH₂CF₂ COOEt with the formation of the new signal at −117 ppm, attributed to the new preterminal CH₂CF₂CH₂OH.

The reaction was considered completed when the conversion of the carboxylic ester was >98%.

The reaction mixture was then let to warm to room temperatures and added with 90 g of an aqueous solution of HCl (10%) and let to stir for 1 hour. The resulting mixture was then poured in a separator funnel and the lower phase was separated and subjected to distillation, thereby affording 75 g F(CH₂CF₂CF₂ O)₆CH₂CF₂ CH₂OH.

### Example 3

### Synthesis of F(CH₂CF₂CF₂O)₆CH₂CF₂CH₂OCH₂CH₂OH

A 100 ml, 3-necked flask, equipped with a magnetic stirrer, a thermometer and a condenser was charged with 30 g F(CH₂CF₂CF₂ O)₆CH₂CF₂CH₂ OH (Mw=875), obtained according to example 2, 5 g of ethylene carbonate (Mw=88) and 0.4 g tBuOK. The resulting solution was then warmed up to 150° C. and let to stir for 15 hours.

The conversion of the reaction was checked by ¹⁹F-NMR analysis, monitoring the disappearance of the preterminal CH₂CF₂CH₂OH at −117 ppm with the formation of a new signal at −115 ppm, attributed to the new preterminal CH₂CF₂CH₂O CH₂CH₂OH. The reaction was considered completed when the conversion of the alcohol was >98%.

The reaction mixture was then let to cool to room temperature and added with 20 g of an aqueous solution of HCl (10%), and let to stir for 1 hour. The resulting mixture was then poured in a separator funnel and the lower phase was separated and subjected to distillation, thereby affording 31 g F(CH₂CF₂CF₂ O)₆CH₂CF₂CH₂OCH₂CH₂OH.

### Example 4

### Synthesis of F(CH₂CF₂CF₂O)₆CH₂CF₂CH₂OCH₂CH_{2 OP(O)(OH)}₂

A 100 ml, 3-necked flask, equipped with a magnetic stirrer, a thermometer and a condenser was charged with 50 g POCl₃ (Mw=152) and warmed up to 60° C.

25 g F(CH₂CF₂CF₂O)₆CH₂CF₂CH₂OCH₂CH₂OH, (Mw=919), obtained according to example 3, was then slowly added and the reaction mixture was let to stir for 2 hours.

The excess POCl₃ was then removed by distillation and the residue was then let to warm to room temperature and added with 30g H₂O. The resulting mixture was then let to stir for 1 hour and poured in a separator funnel; the lower phase was separated and subjected to distillation, thereby affording 26 g F(CH₂CF₂CF₂O)₆CH₂CF₂CH₂OCH₂CH₂OP(O)(OH)₂.

### Example 5

### Synthesis of

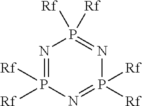

in which R_{f} is F(CH₂CF₂CF₂O)₆CH₂CF₂CH₂O—, in 9:1 molar mixture with

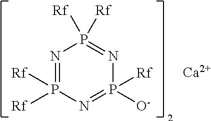

A 500 ml 4-necked flask, equipped with a mechanical stirrer, a thermometer and a condenser was charged with 50 g of a mixture of perfluoropropyl tetrahydropyrane and perfluorobutyltetrahydrofurane as solvent, 40 g F(CH₂CF₂CF₂ O)₆CH₂CF₂CH₂OH (Mw=875), obtained according to example 4, 0.6 g of an aqueous solution at 5% by weight of Bu₄N⁺OH⁻, 2.7g hexachlorocyclotriphosphazene and 50 g of an acqueous solution of KOH at 30% by weight. The resulting mixture was then heated to 60° C. and kept under stirring for about 12 hours, then cooled to allow the formation of an aqueous and of a heavy organic phase. The latter was washed with 50 g water, separated again and washed a second time with 60 g of an aqueous HCl solution at 10% by weight. The organic phase was recovered and the solvent was removed by distillation and the distillation product was added with 2 g of Ca(OH)₂, 5 g H₂O and let to stir at 40° C. for 4 hours. The obtained dispersion was then filtered on a 0.2 μm filter and dried under vacuum (0.1 mmHg) at 60° C. to afford 39 g title product.

1. A hydroxy compound of formula (I)
F(CH₂CF₂CF₂O)ₙCH₂CF₂CH₂OH (I)
wherein n is an integer equal to or higher than 1.

2. A compound selected from:
a) a compound formula IIa)
F(CH₂CF₂CF₂O)ₙCH₂CF₂CH₂OR¹ (IIa)
wherein n is an integer equal to or higher than 1 and R¹ is a (CHR²CHR³O )ₘ−R⁴ group, in which:
R² and R³ are both hydrogen or one is hydrogen and the other is methyl;
m is 0 or an integer higher than 1; and
R⁴ is selected from:
hydrogen, with the proviso that, when m is 0, R⁴ is not hydrogen;
a straight or branched, saturated or unsaturated C₁-C₃₆ hydrocarbon chain;
a P(O)R⁵R⁶ group, in which R⁵ and R⁶ are, independently of each other, selected from hydroxy and —O⁻X⁺ groups, in which X⁺ is selected from Li⁺, Na⁺, K⁺, (NH₃R′)⁺, (NH₂R′R″)⁺ and (NHR′R″R′″) wherein R is H or a linear or branched C₁-C₂₂ alkyl group optionally containing one or more —OH groups, and R′, R″ and R′″, equal to or different from each other, are linear or branched C₁-C₂₂ alkyl groups optionally containing one or more —OH groups or optionally linked to each other to form N-heterocyclic groups;
a COR⁷ group, in which R⁷ is a straight or branched, saturated or unsaturated C₁-C₃₆ hydrocarbon chain;
a CONHR⁸ group, in which R⁸ is the same as R⁷ or R⁸ is a COR⁷ group, in which R⁷ is as defined above; and
a CO—R^{A}—CR_{H}═CH₂ group, wherein R_{H} is hydrogen or a straight or branched, saturated or unsaturated C₁-C₃₄ hydrocarbon chain and R^{A} is selected from the group consisting of
(j) NH—R^{B}—O—CO—,
(jj) NH—R^{B}—NHCOO—R^{B}—OCO— and
(jjj) R^{B}—O—CO—;
R^{B} being a divalent group selected from the group consisting of a C₁-C₁₀ aliphatic group, a C₅-C₁₄ cycloaliphatic group; C₆-C₁₄ aromatic or C₁-C₁₀ aliphaticC₆-C₁₄ aromatic group; and
b) a compound of formula (IIb) 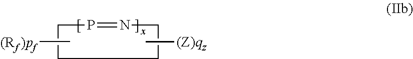
wherein:
R_{f} is a group of formula F(CH₂CF₂CF₂O)ₙCH₂CF₂CH₂O(CHR²CHR³O ),ₘ in which R², R³, n and m are as defined above;
Z is a polar group of formula —O⁻M⁺, wherein M is selected from hydrogen, a monovalent metal, an ammonium radical selected from NRₐR_{b}R_{c}R_{d}, wherein each of Rₐ, R_{b}, R_{c} and R_{d} is, independently, a hydrogen atom or a C₁-C₁₂ hydrocarbon group, optionally fluorinated, or a polar group of formula —O⁻)₂M′²⁺, wherein M′ is a divalent metal, preferably an alkaline earth metal selected from Ca and Mg; and
x is 3 or 4, with the proviso that:
when x is 3, p_{f} is an integer from 1 to 6, q_{z} is 0 or an integer from 1 to 5 and p_{f}+q_{z} is 6 and
when x is 4, p_{f} is an integer from 1 to 8, q_{z} is 0 or an integer from 1 to 7 and p_{f}+q_{z} is 8.

3. A process for preparing a hydroxy compound of formula (I):
F(CH₂CF₂CF₂O)ₙCH₂CF₂CH₂OH (I)
wherein n is an integer equal to or higher than 1, wherein the process comprises:
a) ring-opening a 2,2,3,3-tetrafluorooxethane of formula (III) 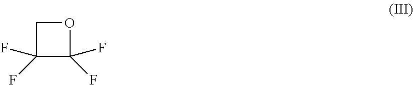
with a metal fluoride to provide a fluoroacyl compound of formula (IV)
F(CH₂CF₂CF₂)ₙCH₂CF₂COF (IV)
wherein n is as defined above;
b) reducing the fluoroacyl compound of formula (IV) to a hydroxy compound of formula (I).

4. A process according to claim 3 in which step b) comprises reacting compound (IV) with an alcohol, to provide an ester that is subsequently reduced to the hydroxy compound of formula (I).

5-6. (canceled)

7. A water and oil resistance compositions comprising a compound of formula (IIa) or (IIb) as defined in claim 2 and at least one additive or vehicle.

8. A compound according to claim 2, wherein the compound is selected from compounds of formula (IIa) and wherein R⁷ is a —CR_{H}═CH₂ group, and R_{H} is selected from hydrogen and straight or branched, saturated or unsaturated C₁-C₃₄ hydrocarbons.

9. A compound according to claim 2, wherein the compound is selected from compounds of formula (IIa) and wherein n is an integer from 1 to 100.

10. A compound according to claim 2, wherein the compound is selected from compounds of formula (IIa) and wherein m is an integer from 0 to 10.

11. A compound according to claim 2, wherein the compound is selected from compounds of formula (IIa) and wherein R² and R³ are both hydrogen.

12. A compound according to claim 2, wherein the compound is selected from compounds of formula (IIa) and wherein R⁴ is hydrogen or a hydrocarbon chain, m is an integer from 0 to 3, and R² and R³ are both hydrogen.

13. A compound according to claim 2, wherein the compound is selected from compounds of formula (IIa) and wherein R⁴ is a P(O)R⁵R⁶ group, m is an integer from 0 to 3, R² and R³ are both hydrogen and R⁵ and R⁶ are both hydroxy groups.

14. A compound according to claim 2, wherein the compound is selected from compounds of formula (IIa) and wherein R⁴ is a COR⁷ group, R⁷ is —C(CH₃)═CH₂, n is an integer from 5 to 15, R² and R³ are both hydrogen and m is an integer from 0 to 3.

15. A compound according to claim 2, wherein the compound is selected from compounds of formula (IIa) and wherein R⁴ is a CONHR⁸ group, R⁸ is —CR_{H}—CH₂ or a COR⁷ group, R_{H} is hydrogen or a straight or branched, saturated or unsaturated C₁-C₃₄ hydrocarbon chain, R⁷ is —C(CH₃)═CH₂, n is an integer from 5 to 15, R² and R³ are both hydrogen and m is an integer from 0 to 3.

16. A compound according to claim 2, wherein the compound is selected from compounds of formula (IIa) and wherein R⁴ is a CO—R^{A}—C(CH₃)═CH₂ group, n is an integer from 5 to 15, R² and R³ are both hydrogen and m is an integer from 0 to 3.

17. A compound according to claim 2, wherein the compound is selected from compounds of formula (IIb) and wherein M is selected from Li, Na and K.

18. A compound according to claim 2, wherein the compound is selected from compounds of formula (IIb) and wherein x is 3, R_{f} is F(CH₂CF₂CF₂O)ₙCH₂CF₂CH₂O(CHR₂CHR₃O)ₘ-, n is an integer from 5 to 15, m is 0 and p_{f} is 6.

19. A compound according to claim 2, wherein the compound is selected from compounds of formula (IIb) and wherein x is 3, R_{f} is F(CH₂CF₂CF₂O)₆CH₂CF₂CH₂O— and p_{f} is 6.

20. A process for preparing the compound according to claim 2, the process comprising:
preparing, as an intermediate, a hydroxy compound of formula (I)
F(CH₂CF₂CF₂O)ₙCH₂CF₂CH₂OH (I)
wherein n is an integer equal to or higher than 1; and
reacting the intermediate under suitable conditions to form a compound of formula (IIa) or a compound of formula (IIb).

21. A method for imparting water and oil resistance to a material, the method comprising contacting the material with a compound according to claim 2.
